(19) United States
(12) Patent Application Publication
(10) Pub. No.: US 2019/0175883 A1
(43) Pub. Date: Jun. 13, 2019

(54) CONDUIT TO INCREASE CORONARY BLOOD FLOW

(71) Applicant: The Trustees of Columbia University in the City of New York, New York, NY (US)

(72) Inventors: Jeffrey D. Wessler, New York, NY (US); Jeffrey W. Kysar, New York, NY (US)

(21) Appl. No.: 16/276,291

(22) Filed: Feb. 14, 2019

Related U.S. Application Data
(63) Continuation of application No. PCT/US2017/047001, filed on Aug. 15, 2017.
(60) Provisional application No. 62/375,063, filed on Aug. 15, 2016.

(51) Int. Cl. A61M 27/00; A61M 39/24

(52) U.S. Cl. CPC A61M 27/002; A61M 39/24; A61M 2039/226; A61M 2205/3303; A61M 2210/125

(57) ABSTRACT

Disclosed is a conduit that is adapted to be positioned in the myocardium to provide a passage for blood to flow from a heart chamber to a coronary artery. The conduit has a sphincter to open or close the passage in response to an electric current applied from a control unit and a one-way valve positioned therein to prevent the backflow of blood from the coronary artery into the heart chamber. Also disclosed is an implantable system or device comprising the conduit, a sensor unit to measure at least one physiological condition and a control unit to analyze signals form the sensor unit and open the sphincter for improving coronary blood flow in order to increase myocardial oxygen delivery during periods of high myocardial demand.

### CROSS-REFERENCE TO RELATED APPLICATIONS

The present application is a continuation of International Application PCT/US2017/047001, filed on Aug. 15, 2017, which claims priority to U.S. Provisional Application No. 62/375,063, filed on Aug. 15, 2016, both of which are herein incorporated by reference in their entirety.

### FIELD OF THE DISCLOSED SUBJECT MATTER

Provided is a system, device and method for improving coronary blood flow and to increase myocardial oxygen delivery during periods of high myocardial demand.

### BACKGROUND

In the era of improved treatment of coronary disease with percutaneous interventions, heart failure (HF) has become a major burden of cardiovascular disease in the developed world. There are currently over 5 million patients with HF in the United States, with 400,000 new diagnoses yearly. Chronic HF is a syndrome that progresses from patients at risk but without structural disease (Stage A) to endstage HF with structural cardiac abnormalities, debilitating symptoms, and high mortality rates (Stage D).

As a patient's HF syndrome progresses, the heart compensates in different ways, including activation of sympathetic nervous system (SNS) that causes the heart rate to increase, and activation of the Renin Angiotensin Aldosterone System (RAAS) that increases preload filling of the ventricles. This leads to ventricular hypertrophy, as the heart pumps faster and more forcefully to augment stroke volume and cardiac output, and ultimately ventricular dilatation, as the heart walls become thinner toward endstage HF. Acute decompensated heart failure (ADHF) occurs when the heart is no longer able to adequately pump blood forward to the rest of the body, causing a buildup of blood volume backwards into the lungs and venous system. ADHF is the main cause of morbidity for HF patients, and leads to increasing hospitalization as a patient's HF syndrome progresses.

The heart has very high energy demands that are increased as HF progresses and structural changes occur. The substrates (oxygen dependent) that support these energy demands are delivered to the heart muscle (myocardium) via coronary circulation. Oxygen reaches the myocardium through coronary arteries and microcirculation. Normally the myocardium extracts a very high ratio of the available blood oxygen content, thus, the main mechanism by which oxygen delivery is increased is by increasing coronary blood flow (CBF). During, and just prior to, periods of ADHF, the myocardium experiences strain with subsequent decreased oxygen supply and increased oxygen demand.

The current standard of care for treatment of HF includes lifestyle recommendations, pharmacologic treatments, invasive implanted devices for specialized populations, and ultimately heart transplant for eligible endstage patients. One type of common drug therapy is administration of vasodilators (hydralazine, nitrates, and ACE inhibitors). Vasodilators increase the diameter of blood vessels in the body and as a result, reduce the strain on the heart. However, this drug therapy decreases blood pressure and could cause hypotension long term. Diuretics are used to reduce the fluid buildup common to heart failure, but do not prevent disease progression or reduce mortality, and are limited by hypotension as well. Angiotensin converting enzyme (ACE) inhibitors, angiotensin II receptor blockers, and beta blockers, are effective in reducing the dangerous cardiac remodeling that occurs as HF progresses, but have limitations in the treatment of endstage HF.

In the late stages of heart failure, devices can be used to treat some consequences of heart failure. A ventricular assist device (VAD) is sometimes implanted to take over the pumping function of the ventricles. Although the VAD restores cardiac output and should eliminate all symptoms associated with heart failure, it does not treat the ischemic cardiac tissue, it merely replaces its function, and is associated with numerous serious complications. Another common type of device is a Cardiac Resynchronization Therapy (CRT) device, which include pacemakers, defibrillators or a combination of both. These devices are used if the contraction of the left and right ventricles is uncoordinated or abnormally fast, and to prevent sudden cardiac death in a subset of qualifying HF patients. Finally, endstage HF can be treated (and cured) with heart transplantation, however this strategy is severely hindered by a limited organ supply and extensive post-transplant complications.

Both pharmacological and device therapies treat the symptoms and physiological results of HF, including decreased cardiac output, fluid retention, increased heart rate and irregular cardiac rhythms. However, few of these therapies are aimed at preventing the progression of HF by modulating underlying pathophysiologic mechanisms behind acute decompensation. Current therapies address specific aspects of HF pathophysiology but have been limited in their capacity to prevent cardiac remodeling and slow progression of HF stages. One aspect of this pathophysiology that has gone largely unmet is improving coronary blood flow (CBF).

Currently, the main mechanism for increasing CBF in chronic and some acute decompensated HF states is via hydralazine plus nitrate medications. Evidence of benefit is limited, however, and has been predominantly found in black populations only. There are currently no device therapies aimed at mechanically augmenting CBF in order to increase myocardial oxygen delivery during periods of high myocardial demand in decompensated HF states. Notably, while coronary stents are used to augment coronary blood flow (CBF), they can only be utilized to bypass areas of blockage (as in coronary artery disease) and at their maximum effectiveness, can only return coronary blood flow to a baseline state, without the ability to improve CBF beyond baseline to levels that would assist the decompensated myocardium. It is therefore desirable to develop alternative ways to increase CBF, especially during periods of high myocardial demand in decompensated HF states.

### SUMMARY

In one aspect, an implantable system and device that target the oxygen supply and demand mismatch during periods of high myocardial strain are provided. Also provided is a left ventricular coronary conduit to increase coronary blood flow in heart failure patients.

In one aspect, an implantable system is provided. The system includes a conduit that connects the left ventricle to the coronary artery. The conduit includes a sphincter and a passive valve, and a sensor unit capable of detecting one or more biological signals. The signals may be arterial blood pressure, intraventricular pressure, blood flow, volume, temperature, oxygen, electrocardiogram, myocardial strain, serum laboratory levels, or another signal. The sensor is operatively connected to a control unit, and transmits data signals relating to the biological signals to the control unit. The control unit may have a memory to record input signals relating to the biological signals from the sensor, and a processor for continuously analyzing the signals received from the sensor and logic to determine whether the sphincter should remain closed or open. The control unit is configured to receive data from the sensor, and actuate opening or closing of the sphincter based on the analyzed data.

In another aspect, a medical device is provided. The medical device is an implantable conduit comprising a tube providing a pathway for blood flow that is adapted to be positioned in the heart wall. A lumen is defined by the interior circumferential wall of the tubular member. The conduit has a proximal end and a distal end defining a length. A sphincter is positioned in the lumen of the tube body. The sphincter includes a central hub section and a plurality of spokes, each of which is connected at a first end to the central hub and at an opposing second end to the interior circumferential wall of the tube. The sphincter is capable of opening and closing the conduit pathway. The conduit may further include a passive one-way valve positioned in the tubular member spaced apart from the sphincter.

In some embodiments, the sphincter is in electric communication with a power supply and is configured to remain closed in the absence of electric current and open when an electric current is applied to it, allowing blood to flow through the tubular member. When a passive one-way valve is included, the passive one-way valve is configured to open when blood flows through the open sphincter from the proximal end of the tubular member and close when blood backflows through the distal end of the tubular member.

Also provided is a method to increase coronary blood flow in a heart failure patient, comprising implanting a device described above into the heart failure patient; detecting one or more biological signals selected from the group including arterial blood pressure, intraventricular pressure, blood flow, volume, temperature, oxygen, electrocardiogram, myocardial strain, serum laboratory levels, or another signal; analyzing the one or more biological signals to determine that increased myocardial demand is present; and applying current to the sphincter to open the sphincter, opening a passageway through the conduit and thereby increasing coronary blood flow.

Notably, analyzing the one or more biological signals and determining that increased myocardial demand is present are conducted via a processor configured to execute computer readable instructions in a control unit of the device, and applying current to the sphincter is via an electric circuit that is actuated by the control unit based on the determination via the processor that increased myocardial demand is present.

### BRIEF DESCRIPTION OF THE DRAWINGS

FIG. 1 shows a schematic view of the implantable system described herein, implanted in a patient.

FIGS. 2A and 2B show schematic views of the conduit described herein in the open and closed configurations respectively.

FIG. 3 shows a schematic view of the sphincter described herein.

### DETAILED DESCRIPTION

Described herein are devices and systems for providing an implantable conduit to allow communication of fluids from one portion of a patient's body to another; and, more particularly, to a blood flow conduit to allow communication from a heart chamber to a vessel or vice versa, and/or vessel to vessel. Even more particularly, the subject matter embodied herein relates to a left ventricular conduit and related conduit configurations for controlling the flow of blood through the conduit to achieve controllable blood flow from the left ventricle to a coronary artery of a patient suffering symptoms of heart failure.

While methods, systems and devices are described herein by way of examples and embodiments, those skilled in the art recognize that the methods, systems and devices for the implantable conduit are not limited to the embodiments or drawings described. It should be understood that the drawings and description are not intended to be limited to the particular form disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “may” is used in a permissive sense (i.e., meaning having the potential to) rather than the mandatory sense (i.e., meaning must). Similarly, the words “include,” “including,” and “includes” mean including, but not limited to.

As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation may not be necessary for using the devices or achieving the methods described herein.

As used herein, the terms “conduit”, “hollow implant,” “tube,” “tubular member” and related terms refer to physical structures, preferably primarily artificial, configured to be implanted in a patient's body, such as positioned between two or more chambers or vessels, that allows the flow of fluid, in particular blood, therethrough, such as blood flow from one chamber or vessel to another. A “shunt” is any natural or artificial passage between natural channels, such as heart chambers or blood vessels. The conduit described herein has control mechanisms including a sphincter and a one-way valve that regulate the flow of fluid through the conduit. As used herein, the “proximal portion” of the conduit is intended to be implanted in the patient's body so that it is in contact and fluid communication with the left ventricle and allows blood to enter the conduit therefrom. The “distal portion” of the conduit is intended to be implanted in the patient's body so that it is in contact and fluid communication with the coronary artery and blood exits the conduit thereto.

As used herein, the term “heart chamber” primarily refers to the interior, or lumenal, aspect of the left or right ventricle or the left or right atrium. The term “heart wall” comprises any one or more of the following portions or layers of the mammalian heart: the epicardium, myocardium, endocardium, pericardium, interatrial septum, and interventricular septum.

The principles of the devices and systems described herein are not limited to left ventricular conduits, and include conduits for communicating bodily fluids from any space within a patient to another space within a patient, including any mammal. Furthermore, such fluid communication through the conduits is not limited to any particular direction of flow and can be antegrade or retrograde with respect to the normal flow of fluid. Moreover, the conduits may communicate between a bodily space and a vessel or from one vessel to another vessel (such as an artery to a vein or vice versa). Moreover, the conduits can reside in a single bodily space so as to communicate fluids from one portion of the space to another. For example, the conduits can be used to achieve a bypass within a single vessel, such as communicating blood from a proximal portion of an occluded coronary artery to a more distal portion of that same coronary artery.

In addition, the conduits and related methods can preferably traverse various intermediate destinations and are not limited to any particular flow sequence. For example, in one preferred embodiment, the conduit communicates from the left ventricle, through the myocardium, into the pericardial space, and then into the coronary artery. However, other preferred embodiments are disclosed, including direct transmyocardial communication from a left ventricle, through the myocardium and into the coronary artery. Thus, as emphasized above, the term “transmyocardial” should not be narrowly construed in connection with the preferred fluid communication conduits, and other nonmyocardial and even noncardiac fluid communication are preferred as well. With respect to the walls of the heart (and more specifically the term “heart wall”), the preferred conduits and related methods are capable of fluid communication through all such walls including, without limitation, the pericardium, epicardium, myocardium, endocardium, septum, etc.

The bypass which is achieved by the preferred embodiments and related methods described herein is not limited to a complete bypass of bodily fluid flow, but can also include a partial bypass or particularly a controllable bypass which advantageously supplements the normal bodily blood flow, such as during episodes of high myocardial strain.

The preferred conduits and related methods disclosed herein can also provide complete passages or partial passages through bodily tissues. In this regard, the conduits can comprise shunts, or the like, and therefore provide a passageway or opening for bodily fluid such as blood. Moreover, the conduits are not necessarily stented or lined with a device but can comprise mere tunnels or openings formed in the tissues of the patient.

The conduits preferably comprise both integral or one-piece conduits as well as plural sections joined together to form a continuous conduit. In order to restore the flow of oxygenated blood through the coronary artery, the preferred arrangement provides for the shunting of blood directly from the heart to the coronary artery.

Although the specification herein will describe the conduit primarily with reference to the left ventricle, the preferred arrangement can be used with any of the four heart chambers, and with any coronary artery, including the left main coronary artery, the right coronary artery, the left anterior descending artery, the left circumflex artery, the posterior descending artery, the obtuse marginal branch or a diagonal branch.

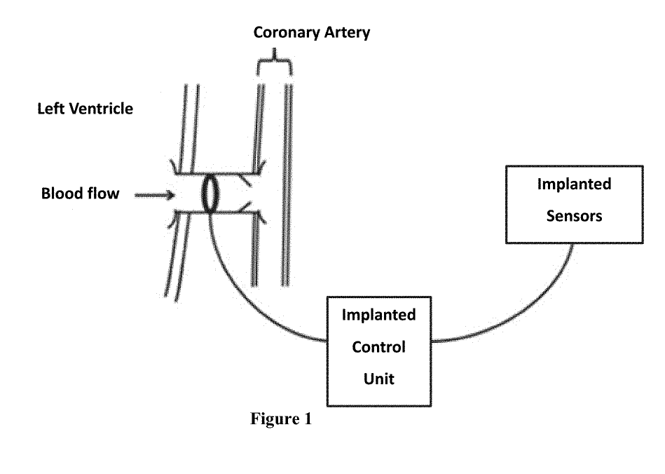

Referring to FIG. 1, the system or device described herein comprises: (i) a conduit comprising a tube that connects the left ventricle to the coronary artery such that blood may flow through the inner lumen of the tube; (ii) a sensor unit; and (iii) a control unit. FIG. 1 shows a schematic view of the implantable system implanted in a patient. A coronary artery bypass is accomplished by disposing the conduit in a heart wall or myocardium of a patient's heart. The conduit is a tube that preferably extends from the left ventricle of the heart to a coronary artery to create a passageway therethrough. The conduit is operatively connected to a control unit, which is also operatively connected to a sensor unit.

The conduit may be placed either via a percutaneous (interventional) approach or surgically. Placement of the device will utilize current catheterization or surgical technologies for the implantation of percutaneous stents, grafts and valves which are in widespread use in interventional cardiology and cardiothoracic surgical practices.

The conduit includes an active valve or sphincter that allows for control of blood flow to the coronary artery. The sphincter may be controlled by an electrical current emitted from the control unit that is programmed to detect when the heart or portion thereof will require more oxygen, for example a portion of the heart supplied from a specific coronary artery. This innovation allows for controlled increased oxygen supply to cardiac tissue, providing the ability to reverse cardiomyopathies caused by heart failure and to reduce or eliminate all heart failure symptoms.

The conduit (i) has three components: a sphincter, a passive one-way valve, and an open-ended tubular member providing a lumen around the sphincter and valve. The components of the conduit (i) will be described in greater detail below.

The sensor unit (ii) is adapted to detect one or several biological signals of physiological conditions such as arterial blood pressure, intraventricular pressure, blood flow, volume, temperature, oxygen, electrocardiogram, myocardial strain, serum laboratory levels, or another signal. Sensor(s) will be positioned within the patient's body at location(s) suitable for monitoring the desired physiological conditions. Notably, the sensor unit is configured to monitor physiological condition(s) that indicate high myocardial demand. The sensor unit is operatively connected to the control unit (iii) and is adapted to send the signal information as input to the control unit.

In some embodiments, the sensor unit may comprise a plurality of sensors. The sensor(s) may be advantageously operatively connected to the sensor unit by sensor leads, allowing the signal(s) from the sensors to be aggregated in a central sensor unit. The central sensor unit may be housed and positioned in the patient separately from the control unit. Alternatively, the central sensor unit and the control unit may be positioned in proximity to each other and housed in a single casing.

The control unit (iii) has a memory to record input signals relating to the biological signals from the sensor unit (ii); a processor for continuously analyzing the signals received from the sensor and logic to determine whether a physiological condition indicating myocardial strain is present. Based on the analyzed data, the control unit will determine whether the sphincter should remain closed or be opened in response to indications of myocardial strain. The control unit is configured to actuate opening or closing of the sphincter based on the analyzed data. For example, a current will be supplied to the sphincter to open it or current will not be supplied to the sphincter to close it.

The control unit will also contain a battery to power its operation, supply power to the sensor unit and to the sphincter and, along with all input and output signal connections, will be enclosed by a casing material and coated in antithrombotic or antistenotic agents. Titanium is a preferred casing material for the casing of the control unit, such as commonly used in pacemakers.

Additional iterations of this device may include multiple conduits and sphincters to be stimulated at the same time or sequentially.

The device will be placed either via a percutaneous (interventional) approach or surgically. It is similar to devices that have been utilized in the past two decades to simulate coronary artery bypass grafting (CABG) for coronary disease—most of which have been abandoned in the setting of vast improvements in stent technologies that make the need for this conduit for coronary disease obsolete. The concept is now being repurposed for the treatment of heart failure, with the addition of a signal-controlled and activated sphincter that renders the device active in situations of high myocardial demand. Placement of the device will utilize current catheterization or surgical technologies for the implantation of percutaneous stents, grafts and valves which are in widespread use in interventional cardiology and cardiothoracic surgical practices.

In some embodiments the control unit and/or the sensor unit, or a casing comprising both the control unit and the sensor unit may be positioned inside the chest cavity, with leads operatively connected to the sphincter and the sensor(s) respectively. Alternatively, the control unit and/or the sensor unit, or a casing comprising both the control unit and the sensor unit may be placed subcutaneously outside the chest cavity with leads operatively connected to the sphincter and the sensor(s) respectively. Placement outside the chest cavity may allow access to the control unit and/or the sensor unit without the need for invasive procedures. Access may be needed for maintenance, replacement of components such as the processor or battery, or to provide operative connection to download data or update software associated with the device.

The system or device provides a conduit or shunt for diverting blood directly from the left ventricle of the heart to a coronary artery. The conduit preferably comprises a tube adapted to be positioned in the myocardium and having a sphincter and one-way valve therein. The sphincter provides a mechanism for opening or closing the conduit in response to an electrical signal. The one-way valve prevents the backflow of blood from the coronary artery into the left ventricle.

As is well known, the coronary artery branches off the aorta and is positioned along the external surface of the heart wall. Oxygenated blood that has returned from the lungs to the heart then flows from the heart to the aorta. Some blood in the aorta flows into the coronary arteries, and the remainder of blood in the aorta flows on to the remainder of the body. The coronary arteries are the primary blood supply to the heart muscle and are thus critical to life. The present conduit provides the ability to controllably increase oxygenated blood flow into the coronary artery and prevent damage to the heart during periods of high myocardial strain.

A tunnel or opening is formed through the wall of the coronary artery and the heart wall and into the left ventricle of the heart which lies beneath, or deep to, the coronary artery. A conduit as described herein is positioned in the opening to keep it open, with control measures to regulate blood flow therethrough.

The conduit may be introduced into the heart wall in a variety of ways, including by a catheter threaded through the femoral artery into the aorta and thence into the left ventricle and, if necessary, the left atrium; or by a catheter threaded through the femoral vein into the inferior vena cava and thence into the right atrium and right ventricle. Alternatively, the conduit may be introduced through a surgical incision in chest wall (thoracotomy) or sternum (sternotomy).

The opening through the heart wall (including endocardium, myocardium, and epicardium) and coronary artery can be formed in a variety of ways, including by knife or scalpel, electrocautery, cryoablation, radiofrequency ablation, ultrasonic ablation, and the like. Other methods will be apparent to those of ordinary skill in the art. The opening forms a passage through the myocardium, with a free edge at each margin of the cut or puncture. The conduit is inserted into the opening so the proximal end of the conduit contacts the free edges of the myocardium and provides a permanent passageway through the myocardium.

The present conduit can be deployed in a variety of methods consistent with sound medical practice including vascular or surgical deliveries, including minimally invasive techniques. For example, various preferred embodiments of delivery rods and associated methods are disclosed. In one embodiment, the delivery rod is solid and trocar-like. It may be rigid or semi-rigid and capable of penetrating the tissues of the patient and thereby form the conduit, in whole or in part, for purposes of fluid communication. In other preferred embodiments, the delivery rods may be hollow so as to form the conduits themselves (e.g., the conduits are preferably self-implanting or self-inserting) or have a conduit mounted thereon (e.g., the delivery rod is preferably withdrawn leaving the conduit installed). Thus, the preferred conduit device and method for installation is preferably determined by appropriate patient indications in accordance with sound medical practices.

Preferably, the conduit device is delivered to a position in the left ventricle in close proximity to the coronary artery. At that position, the coronary artery, the myocardium and the wall of the left ventricle are pierced to provide an opening or channel completely through from the left ventricle of the heart to the coronary artery. The conduit is then positioned in the opening to provide a permanent passage for blood to flow from the left ventricle of the heart to the coronary artery. The length of the conduit is sized so that the distal open end is positioned within the coronary artery, while the proximal open end is positioned in the left ventricle. The length of the conduit may be variable, depending on the thicknesses of the heart wall and the coronary artery and the distance between them. The conduit may be fabricated by preparing a proximal portion comprising the sphincter and a distal portion comprising the one-way valve and inserting them into or attaching them to the open ends of a tubular member of a desired length. The hollow lumen of the conduit provides a passage for the flow of blood. The diameter of the conduit may be preferably about 2 mm.

The device wherein the hollow implant is configured to be positioned in the heart wall between a heart chamber and a blood vessel.

The device wherein the hollow implant is configured to be positioned in the heart wall between a left ventricle and a blood vessel.

The device wherein the hollow implant is configured to be positioned in the heart wall between the heart chamber and a coronary artery.

The device wherein the hollow implant is configured to be positioned in the heart wall between a left ventricle and a coronary artery.

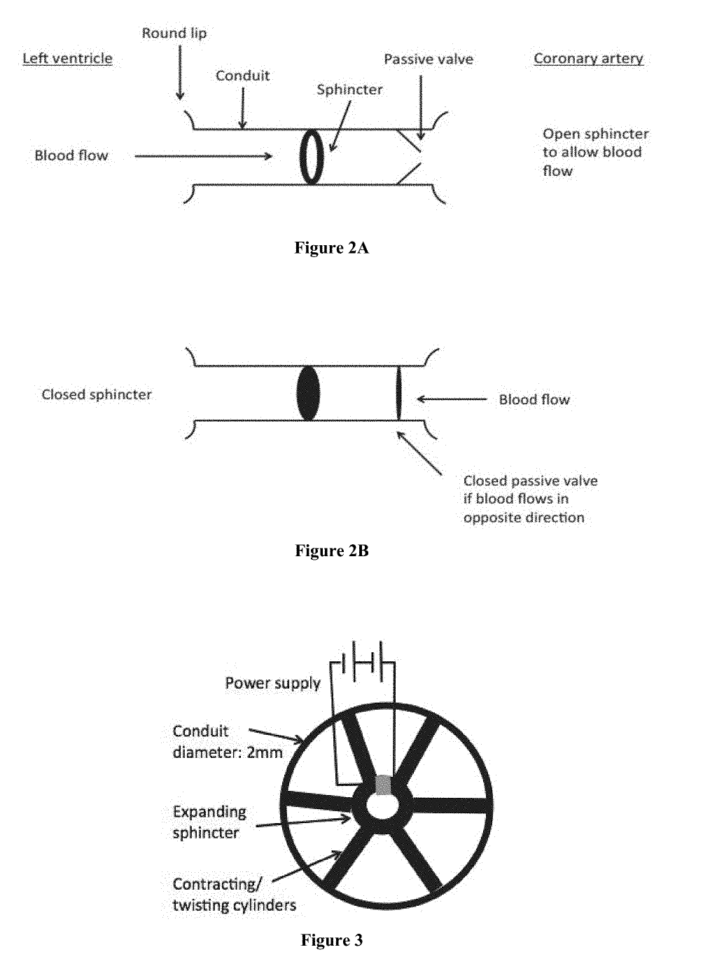

Referring to FIGS. 2A and 2B, the conduit comprises three components: a sphincter, a passive one-way valve, and an open-ended cylinder around the sphincter and valve.

FIGS. 2A and 2B show schematic views of the conduit implanted in the patient's body wherein the proximal end is in fluid communication with the left ventricle and the distal end is in fluid communication with a coronary artery. In FIG. 2A, the sphincter is in the open configuration, and blood can pass through the conduit. High pressure blood flow from the left ventricle, such as during systole, passes through the open sphincter and contacts the passive one-way valve. Pressure in the direction indicated by the arrow causes the one-way valve to open, allowing blood to flow out of the conduit into the coronary artery. When the sphincter is open due to application of electric current from the control unit, blood can flow from the left ventricle through the passageway to the coronary artery. Although the conduit may elastically deform under the contractive pressure of the heart muscle during systole, the passageway remains open to allow blood to pass from the patient's left ventricle into the coronary artery. In FIG. 2B, the sphincter is in the closed configuration, and blood cannot pass through the conduit. When the sphincter is closed, blood does not flow from the left ventricle through the passageway, and the one-way valve is in the closed configuration to minimize back flow from the coronary artery through the passageway. Blood pressure in the coronary artery can cause backflow into the distal end of the conduit, contacting the one-way valve and causing it to close, thereby blocking blood backflow. Not shown is the condition wherein the sphincter is in the open position during diastole, with no blood flowing out of the left ventricle. Blood pressure in the coronary artery causes backflow into the conduit, contacting the one-way valve and causing it to close, thereby blocking blood backflow into the conduit and returning to the left ventricle.

The tubular member surrounding the valve and sphincter may comprise biocompatible metal or polymer material, and may comprise either a solid hollow tube or a lattice of material in the form of a tube. A preferred material for the conduit is stainless steel, commonly used in stents due to its durability and biocompatibility. Another preferred material comprises nitinol, although other materials such as Ti, Ti alloys, Ni alloys, Co alloys and polymers may also be used. The materials may be coated in antithrombotic or antistenotic agents. Preferably, the conduit will have a rounded lip on both the proximal and distal ends for stabilization in the heart wall and the coronary artery respectively after implantation.

The conduit also comprises electrical connectors to operatively link the sphincter to the control unit or to lead(s) from the control unit, forming an electrical circuit.

The sphincter comprises an electroactive material that contracts when current is applied to it from the control unit, opening the sphincter and thus opening the conduit. The control unit supplies current to the sphincter when it determines that a condition of high myocardial strain is present. Blood from the left ventricle is able to pass through the conduit into the coronary artery and increase oxygenated coronary blood flow to the area of the heart supplied by the coronary artery.

When current is not supplied from the control unit (when myocardial strain is low), the electroactive material expands, closing the sphincter and thus closing the conduit and halting blood flow.

Referring to FIG. 3, one embodiment of the sphincter design is a “wheel” model, shown schematically in a cross-section view of the conduit. The sphincter comprises an electrical connector that is in a switchable electric circuit with the power supply in the control unit. The sphincter comprises two materials, a dielectric and an electroactive polymer. When the power supply is on, the electroactive polymer will contract and the dielectric will stretch, opening the diameter of the conduit and allowing blood flow through it. The electroactive material comprises a plurality of helical or corkscrew cylinders. Twisting or contracting cylinders will allow for the diameter enlargement. Spaces between the cylinders will be closed on either end of the conduit to block blood flow through them. Materials for the sphincter and cylinders include silicone elastomer (such as CF19-2186 available from NuSil), polyvinylidene difluoride (PVDF) and poly(vinylidene fluoride-co-trifluoroethylene) (PVDFTrFE). The manufacturing technique for the “wheel” model may be a high aspect ratio molding microfabrication process.

An alternative embodiment of the sphincter comprises a plurality of actuators that function as hinges acting in response to the electrical signals from the control unit to open and close the sphincter. Notably, the actuators comprise electroactive materials. For example, the sphincter can be biased in a closed position. When current is off and the electroactive material is in a relaxed or expanded state, each hinge is in a configuration that extends across a portion of the cross section of the conduit to block flow. When the control unit signals the actuator by application of an electric current, the electroactive material in the hinge contracts and causes the hinge to bend toward the inner surface of the conduit. In the bent configuration, each hinge rests alongside the surface of the conduit instead of across the conduit, opening the sphincter and allowing for the flow of blood therethrough.

In some embodiments the sphincter is configured to be in a binary open or closed mode, depending on whether current is on or off. Alternatively, the sphincter may be configured to provide a variable amount of opening depending on the amount of current applied. Also, the sphincter may function to completely block blood flow when in the “closed” configuration. Alternatively however, in some embodiments, the sphincter may be configured to allow a low amount of blood to flow through the conduit in a partially closed manner when no current is applied, and a higher amount of blood flow when current is applied.

To prevent the backflow of blood from the coronary artery to the left ventricle of the heart, the conduit is provided with a passive one-way valve. The passive valve will allow blood flow in one direction only; such as only from the left ventricle to the coronary artery. It will not have a power supply and may be similar to existing passive valve technology.

The one-way valve is preferably a windsock type valve, a flapper valve, a bi- or tricuspid valve, a ball valve, or a valve that opens and closes in response to the contraction and relaxation of the heart muscle.

The one-way valve may comprise the following embodiments. The description of how each embodiment of the one-way valve functions are under the condition of the sphincter being open allowing blood to flow from the left ventricle through the conduit. As noted above, when the sphincter is closed, the various embodiments of the one-way valve are in the closed position.

One embodiment of the valve incorporates a design comprising an open tube configured in a truncated cone (similar to a windsock) with the wider end of the tube positioned toward the proximal end of the conduit and the narrow end of the tube positioned toward the distal end of the conduit. The valve is preferably formed from a biocompatible and very compliant fabric or other material and preferably incorporated during the construction of the conduit. The high-pressure blood flow from the left ventricle during systole causes the valve to open, while the backflow of blood from the coronary artery during diastole catches the edges of the narrow end of the valve and causes it to close, stopping the flow.

Another embodiment of the preferred arrangement incorporates a type of “flapper valve” that is built onto the end of the conduit that is positioned in the coronary artery. The high-pressure blood flow from the ventricle opens the flap and the backflow of blood causes the flap to shut. This flap is slightly larger than the conduit inner diameter to accomplish this action and to ensure a proper seal. The valve is preferably formed from the same material as the conduit and the two are preferably introduced as a single unit.

A third embodiment of the valve is similar to a natural heart valve. A bi- or tricuspid arrangement of semi-circular spheres is forced open by the high-pressure flow and collapses back to prevent backflow of blood through the conduit. This valve is preferably made from the same material as the conduit, or alternatively, from a thin biocompatible material that is built onto the conduit. Preferably, the valve and the conduit are manufactured together and introduced as a single unit. Alternatively, the valve may be attached to the conduit in a secondary operation once the conduit is in place.

In another embodiment, the valve in the conduit may be controlled in response to the contractions of the heart. The conduit, having upper moveable components and lower moveable components, is positioned in the passage in the myocardium between the left ventricle and the coronary artery. The conduit contains a valve, which is normally in a closed position, and an actuator, which is adapted to open the valve in the conduit. During diastole, when the heart muscle is relaxed, the two components of the conduit are positioned such that the valve remains closed. During systole with the sphincter open, the two components are brought close together, such that the actuator forces the valve to open and allows for the passage of blood therethrough. Thus, the contractions of the heart muscle control the valve in the conduit to prevent the backflow of blood during part of the cardiac cycle, for example diastole.

Another embodiment comprises a type of “ball valve” that is built into the conduit that is positioned in the coronary artery. The high-pressure blood flow from the left ventricle to the coronary artery opens the valve by moving the ball away from the opening. The backflow of blood from the coronary artery toward the left ventricle causes the ball to seat against the opening, thereby closing the valve and preventing the backflow of blood.

Another embodiment comprises a conduit comprising a valve with one or more spring mechanisms within its walls. In diastole, blood flow pressure through the valve is relatively low, and the valve assumes a relatively closed position, impeding the passage of blood through the valve. In systole, flow pressure through the valve is relatively high, and the valve opens as the spring mechanism contracts, to allow blood to flow through the valve.

Instead of a spring mechanism, the walls of the conduit can have other mechanisms therein to allow differential flow during various parts of the cardiac cycle. For example, the valve can have a gas- or liquid-filled balloon in its wall, which can contract during systole or expand during diastole in response to fluid pressure, to allow the valve to open and close, respectively.

Another embodiment of the valve mechanism comprises a conduit having a ball valve that is of the ball-and-cage variety, for example, like the Starr-Edwards heart valve known to those of skill in the art. This valve typically has a wire or mesh cage with a ball within it. The conduit is positioned within the myocardium. During blood flow from the left ventricle to the coronary artery, the ball moves toward the apex of the cage, permitting blood to flow around the ball and through the conduit. During backflow of blood from the coronary artery to the left ventricle, the ball moves toward the base of the cage and seats thereon, fitting tightly onto the base of the cage, and blocking the flow of blood from the coronary artery to the left ventricle.

In another embodiment a ball is provided within a conduit that is wider at proximal end facing the left ventricle, and narrower at distal end facing the coronary artery. A similar embodiment comprises a conduit having a gradual taper from the proximal end to distal end. During blood flow from the proximal end to distal end, the ball moves toward the coronary artery to allow blood flow around the ball through the conduit. In one embodiment, the cross-section of the conduit is noncircular, for example elliptical, to allow blood to flow around the ball. During backflow from the coronary artery toward the left ventricle, the ball moves against the base of the conduit to block flow of blood therethrough.

The systems, devices and methods described herein provide significant improvements in the treatment of heart failure patients to ameliorate myocardial strain by providing for increased coronary blood flow. Although the system, device and method have been described in their preferred embodiments in connection with the particular figures, it is not intended that this description should be limited in any way.

Having described and illustrated the principles of the systems, devices and methods with reference to the described embodiments, it will be recognized that the described embodiments can be modified in arrangement and detail without departing from such principles. It should be understood that the systems, processes, or methods described herein are not related or limited to any particular type of environment, unless indicated otherwise.

In view of the many possible embodiments to which the principles can be applied, we claim all such embodiments as can come within the scope and spirit of the following claims and equivalents thereto.

1. A system comprising:
a conduit tube having a proximal end and a distal end and a lumen therethrough, the conduit sized suitably for implantation between a left ventricle and a coronary artery of a subject, and providing a blood flow between said left ventricle and coronary artery of said subject, the conduit tube further including a sphincter spaced from a passive one-way valve, wherein the sphincter and passive one way valve are disposed in the lumen;
a sensor unit capable of detecting one or more biological signals from the subject, the sensor unit adapted to send data relating to the detected biological signal;
a control unit operatively connected to the sensor unit and configured to receive data from the sensor unit, the control unit having a memory to record input signals relating to data received from the sensor, and a processor for continuously analyzing the data received from the sensor and logic to determine whether the sphincter should remain closed or open,
further wherein the control unit is configured to actuate opening and closing of the sphincter based on the analyzed data.

2. The system of claim 1 wherein the control unit applies an electric current to open the sphincter when signals from the sensor unit provide information that a physiological condition indicating high myocardial strain is present.

3. The system of claim 1 wherein the sphincter is positioned in the proximal portion of the conduit tube and the passive one-way valve is positioned in the distal portion of the tubular member; wherein the sphincter is in electric communication with a power supply and is configured to remain closed in the absence of electric current and open when an electric current is applied to it, allowing blood to flow through the tubular member; and the passive one-way valve is configured to open when blood flows through the open sphincter from the proximal end of the tubular member and close when blood backflows through the distal end of the tubular member.

4. The system of claim 1, wherein only the conduit is implantable.

5. The system of claim 1, wherein the biological signal is selected from the group consisting of: arterial blood pressure, intraventricular pressure, blood flow, volume, temperature, oxygen, electrocardiogram, myocardial strain, serum laboratory levels, and a combination therefrom.

6. The system of claim 1, wherein the passive one-way valve comprises a windsock type valve, a flapper valve, a bi- or tricuspid valve, a ball valve, or a valve that opens and closes in response to the contraction and relaxation of the heart muscle.

7. A medical device comprising a hollow implant comprising
an elongate tubular member having an interior wall surface defining a lumen comprising a proximal portion and a distal portion; a sphincter positioned in the lumen and spaced from a passive one-way valve positioned in the lumen of the elongate tubular member;
wherein the sphincter is in electric communication with a power supply and is configured to remain closed in the absence of electric current and open when an electric current is applied to it, allowing blood to flow through the elongate tubular member;
and the passive one-way valve is configured to open when blood flows through the open sphincter from the proximal end of the tubular member and close when blood backflows through the distal end of the tubular member.

8. The medical device of claim 7 wherein the passive one-way valve comprises a windsock type valve, a flapper valve, a bi- or tricuspid valve, a ball valve, or a valve that opens and closes in response to the contraction and relaxation of the heart muscle.

9. The medical device of claim 7, wherein the sphincter comprises a central hub and a plurality of spokes extending laterally from the hub to the interior wall surface of the elongate tubular device.

10. A sphincter valve, comprising:
a central hub section and a plurality of elongate spokes having opposing first and second ends, wherein the first end is connected to the central hub, and the central hub section is movable between valve open and valve closed positions.

11. The sphincter valve of claim 9, wherein the sphincter is in electric communication with a power supply.

12. The sphincter valve of claim 10, wherein the central hub is biased in a valve closed position in the absence of electric current and moves to a valve open position when an electric current is applied to it.

13. A conduit comprising:
a tube body having opposing ends and a lumen defined by a circumferential wall, the wall having an interior and exterior,
a sphincter disposed in the lumen of the tube body, wherein the sphincter includes a central hub section and a plurality of elongate spokes having opposing first and second ends, wherein the first end of the spoke is connected to the hub and the second end of the spoke is connected to the interior wall of the tube.

14. The conduit of claim 13, wherein the central hub is biased in a valve closed position in the absence of electric current and moves to a valve open position when an electric current is applied to it.

15. The conduit of claim 14, wherein the central hub is biased in a valve closed position in the absence of electric current and moves to a valve open position when an electric current is applied to it.

16. The conduit of claim 13, wherein the conduit further includes a passive one-way valve disposed in the tube body.

17. The conduit of claim 16, wherein the passive one-way valve is configured to open when blood flows through the open sphincter from the proximal end of the tubular member and close when blood backflows through the distal end of the tubular member.

18. A conduit comprising
a tube body having opposing ends and a lumen defined by a circumferential wall, the wall having an interior and exterior,
a sphincter disposed in the lumen of the tube body, wherein the sphincter incudes a central hub section and a plurality of elongate spokes having opposing first and second ends, wherein the first end of the spoke is connected to the hub and the second end of the spoke is connected to the interior wall of the tube and a passive one-way valve disposed in the tube body.

19. The conduit of claim 18, wherein the central hub is biased in a valve closed position in the absence of electric current and moves to a valve open position when an electric current is applied to it.

20. The conduit of claim 19, wherein the central hub is biased in a valve closed position in the absence of electric current and moves to a valve open position when an electric current is applied to it.
